(19) United States
(12) Patent Application Publication
(10) Pub. No.: US 2016/0120395 A1
(43) Pub. Date: May 5, 2016

(54) DISPOSABLE ENDOSCOPE SHEATH

(71) Applicant: TIANJIN BOLANG SCIENCE-TECHNOLOGY DEVELOPMENT CO. , LTD., Tianjin (CN)

(72) Inventor: Lin QI, Tianjin (CN)

(21) Appl. No.: 14/898,677

(22) PCT Filed: Jun. 28, 2013
(86) PCT No.: PCT/CN2013/078303

§ 371 Date: Dec. 15, 2015

(30) Foreign Application Priority Data
Sep. 25, 2012 (CN) 201220494633.9
Sep. 25, 2012 (CN) 201220496185.6

(51) Int. Cl. A61B 1/00; A61B 1/018; A61B 1/04; A61B 1/015

(52) U.S. Cl. CPC A61B 1/00135; A61B 1/00103; A61B 1/015; A61B 1/018; A61B 1/0014; A61B 1/00066; A61B 1/00073; A61B 1/00114; A61B 1/04

(57) ABSTRACT

The present invention discloses a disposable endoscope sheath, comprising an insertion portion, a control portion and an endoscope locking portion, wherein the insertion portion comprises a sheath head and a sheath tube; the control portion comprises a main body, a water inlet and outlet valve and an instrument adjusting knob; and the endoscope locking portion comprises an extension endoscope bridge, a locking sleeve and a protective cover. The sheath head is located at the front end of the insertion portion and is connected with the sheath tube in the rear. The sheath tube is connected with the extension endoscope bridge of the endoscope locking portion through a main body. The water inlet and outlet valve and the instrument adjusting knob are located on the main body of the endoscope sheath. The sheath tube is a multi-channel structure, and the outer surface of the insertion portion has scale marks. The present invention has the beneficial effects in that the disposal use avoids cross infection, and the demands of different endoscopic surgeries can be satisfied.

### BACKGROUND OF THE INVENTION

1. Technical Field

The present invention belongs to the technical field of medical instruments, in particular, to a disposable endoscope sheath.

2. Description of Related Art

Conventional cystoscope instruments and hysteroscope instruments are mainly made of stainless steel, while the stainless steel surgical instruments belong to open instruments and cannot completely close an endoscope in use. During surgery, the endoscope works while being inserting into instruments like a sheath. Humoral substances generated during the surgery on the human body pollute the endoscope, and the endoscope must be sterilized after the surgery. Currently, a kind of endoscope sheath which performs fully-closed protection is available on the market. The endoscope is inside, while the sheath is outside. The endoscope does not contact with anyone, so it is the endoscope sheath that contacts the human body. The human body does not pollute the endoscope, thus reducing cross infection. In this way, the endoscope is not needed to the sterilized, capable of protecting the endoscope and maximizing the service life of the endoscope. On the other hand, the manufacturing cost of the disposable endoscope sheath is lower than that of the common endoscope sheaths, capable of reducing the economical burden on patients and meeting more surgical needs.

A Chinese patent, authorized publication No. CN100569175C, discloses a disposable cystoscope sheath. This solution provides a two-channel scope sheath structure capable of accommodating the endoscope and the surgical instruments in the human body to perform diagnosis. However, such scope sheath structure also has the problems. First, instruments are currently applied to the endoscopic surgery include varieties of pliers, scissors and other tools. When the instrument is inserted into the human body through the instrument channel, the inserted instrument needs swinging a certain angle due to the different focus locations, and sometimes the instrument needs rotating to meet more surgical demands. Besides, when the endoscope is placed in the endoscope sheath, the endoscope length may be not identical because the manufacturers are different. If the sheath length is not adjustable, when the endoscope is too long, it may penetrate out of the protective structure of the sheath head of the endoscope sheath, causing injury to the human body; when the endoscope is too short, the rear end of the endoscope sheath cannot shrink and lock the endoscope, affecting the use of the endoscope. Besides, the locking structure in this solution can be repeatedly used after the endoscope is dismantled at the end of the surgery, tending to cause cross infection.

At present, during the endoscopic surgery, sometimes it is needed to extract the water from or injected water into the human body, so the water inflow and outflow channel is also needed in the endoscope sheath. As mentioned above, some endoscopic surgeries need to adjust the angle of the surgical instruments. The sheath tube also requires an adjusting channel, for example a steel wire channel. Due to the limited cross section area of the sheath tube of the endoscope sheath, how to rationally locate the above-mentioned channel to meet the different demands of the modern surgeries is also a technical problem in this field. On the other hand, in the endoscopic surgery, sometimes it is difficult for the medical personnel to judge the insertion depth of the sheath tube of the endoscope sheath, so a means for clearly representing the insertion depth of the sheath tube is also needed.

In the current surgeries, sometimes a lot of body fluids may occur because of excessive bleeding of the patient, etc. In such circumstances, the rear end of the main body of the endoscope and the subsequent part in connection with the CCD system may also be polluted by the body fluid, so an endoscope sheath structure capable of protecting the rear end of the main body of the endoscope and the subsequent part in connection with the CCD system as well as the cables are urgently needed to handle of case of massive surge of the body fluid.

### BRIEF SUMMARY OF THE INVENTION

The problem to be solved in the present invention is to provide a disposable endoscope sheath, in particular to meeting the different types of endoscopic surgeries.

To solve the above-mentioned problems, the present invention adopts the following technical solutions.

The present invention discloses a disposable endoscope sheath, comprising an insertion portion, a control portion and an endoscope locking portion; wherein the control portion comprises a sheath head and a sheath tube; the control portion comprises a main body, and a water inlet and outlet valve; the endoscope locking portion comprises an extension endoscope bridge and a locking sleeve; the sheath head is located at the front end of the insertion portion and is connected with the sheath tube in the rear; the sheath tube is connected with the extension endoscope bridge of the endoscope locking portion; the water inlet and outlet valve and an instrument adjusting knob are located on the main body of the endoscope sheath; characterized in that, the sheath tube comprises an endoscope cavity, an instrument channel, a steel wire channel and a water inflow and outflow channel; the lower part of the front end of the sheath tube is provided with a surgical instrument angle adjusting device, and this device may be a fixed projection, a lifting device or a replaceable projection.

Wherein, the locking sleeve comprises an endoscope bridge interface, an endoscope locking sleeve, a locking lifting lever, a locking compression ring and a disposal connecting ring connected to the tail end of the endoscope bridge; the endoscope locking sleeve and the locking compression ring are integrally connected by using a disposable connecting ring; the exterior of the locking compression ring is connected with the locking lifting lever; the shape of the inner hole of the clamping plate of the locking compression ring corresponds to that of the locking plate of the locking interface of the endoscope; the size of the clamping plate of the locking compression ring is matched with a locking groove of the locking interface of the endoscope; an inner hole of the endoscope locking sleeve is provided with a movable one-way gear; the movable one-way gear is provided with screw threads at the rear end; the exterior of the tail end of the endoscope bridge is also provided with one-way gear and screw threads in turn, matched with the movable gear of the inner hole of the endoscope locking sleeve and connected with the endoscope locking sleeve in a threaded way.

Wherein, the lower part of the front end of said endoscope cavity is provided with an inserting groove, flanges on two sides of the inserting groove and a stopper, which is not on the same side with the flanges. The inserting groove is a dovetail groove; the dovetail groove is a trapezoidal dovetail structure; and the inserting groove and the flanges are formed into a replacement groove for replacing the projection.

Wherein, the endoscope sheath also comprises an instrument adjusting knob; the instrument adjusting knob comprising a gear and a rack; the gear and the rack being engaged; one end of a steel wire is fixed on the rack, while the other end being connected with the lifting device via a steel wire channel.

Wherein, the extension endoscope bridge comprises an endoscope bridge, a rebound blade and a groove-shaped space; the rebound blade is disposed inside the endoscope bridge; and the groove-shaped space is located at the rear end of the endoscope sheath.

Wherein, the sheath head is provided with an arc-shaped transparent window; the inner surface of the arc-shaped transparent window is designed to be smooth, and the corresponding central angle is an acute angle.

Wherein, the endoscope cavity of the sheath tube is internally sleeved with a metal tube to prolong the length of the endoscope cavity of the endoscope sheath.

Wherein, the inner hole of the endoscope bridge interface at the tail end of the endoscope bridge is of a conical design adapted to the locking interface of the endoscope.

Wherein, the rear end portion of the endoscope sheath is also provided with a protective cover; one end of the protective cover wraps up the rear end of the main body of the control portion of the endoscope sheath, while the other end wraps up a cable of a CCD observation system.

Wherein, the outer surface of said insertion portion has scale marks; and the scale marks are disposed from the rear end of the sheath head to the main body of the control portion.

Wherein, the materials of said gear and rack are medical plastic materials with high hardness.

Wherein, the extension endoscope bridge also comprises a rubber ring and a rubber sleeve; and said rubber ring and rubber sleeve are made from silica gel.

Wherein, the material of the protective cover is medical watertight plastic.

The present invention has the following advantages and beneficial effects: By using the above solution, the instrument angle is more conveniently to adjust in the surgery, capable of meeting the demands of different endoscopic surgeries. The structure of the extension endoscope bridge can be used by endoscopes of different lengths. The disposable self-destruction type endoscope locking structure can avoid the endoscope sheath from being reused and causing cross infection. The scale marks of the insertion portion can help the medical personnel to know the current insertion depth of the endoscope sheath. The protective cover can avoid the rear end of the endoscope and the CCD observation structure in connection from being polluted by body fluids. Therefore, the present invention has advantages of avoiding cross infection, being easily accepted on the market, along with rational structural layout and convenient use.

### BRIEF DESCRIPTION OF THE SEVERAL VIEWS OF THE DRAWINGS

FIG. 1 is a schematic view of the overall structure of a disposable endoscope sheath;

FIG. 2 is a structural view of the sheath head portion of a disposable endoscope sheath;

FIG. 3 is a partial exploded view of the sheath head portion of a disposable endoscope sheath;

FIGS. 4-7 are schematic views of the cross section of the sheath tube of a disposable endoscope sheath;

FIG. 8 is transversal sectional view of the cross section of the sheath tube of a disposable endoscope sheath;

FIG. 9 is a structural view of the lifting portion of the front end of a disposable endoscope sheath;

FIG. 10 is transversal sectional view of an extension endoscope bridge of a disposable endoscope sheath;

FIG. 11 is an internal structural view of the main body of a control portion of a disposable endoscope sheath;

FIG. 12 is transversal sectional view of a locking sleeve of a disposable endoscope sheath;

FIG. 13 is a structural view of a protective cover portion of a disposable endoscope sheath;

FIG. 14 is a schematic view of a scale mark portion of a disposable endoscope sheath.

In the figures: 1. Sheath head, 2. Sheath tube, 3. Water inlet and outlet valve, 4. Instrument adjusting knob, 5. Main body, 6. Extension endoscope bridge, 7. Locking sleeve, 8. Protective cover, 11. Arc-shaped transparent window, 12. Lifting device, 13. Inserting groove, 14. Flange, 15. Replaceable projection, 21. Endoscope cavity, 22. Instrument channel, 23. Water inflow and outflow channel and instrument channel, 24. Water inflow and outflow channel, 25. Steel wire channel, 26. Metal tube, 27 Scale marks, 41. Rack, 42. Gear, 43. Steel wire, 61. Rebound blade, 62. Rubber ring, 63. Rubber sleeve, 64. Groove-shaped space, 71. Endoscope locking sleeve, 72. Locking lifting lever, 73. Locking, 74. Disposable locking cracking slot, 81. Endoscope rear end, 82. CCD system interface, 83. Video cable, 84. Optical cable

### DETAILED DESCRIPTION OF THE INVENTION

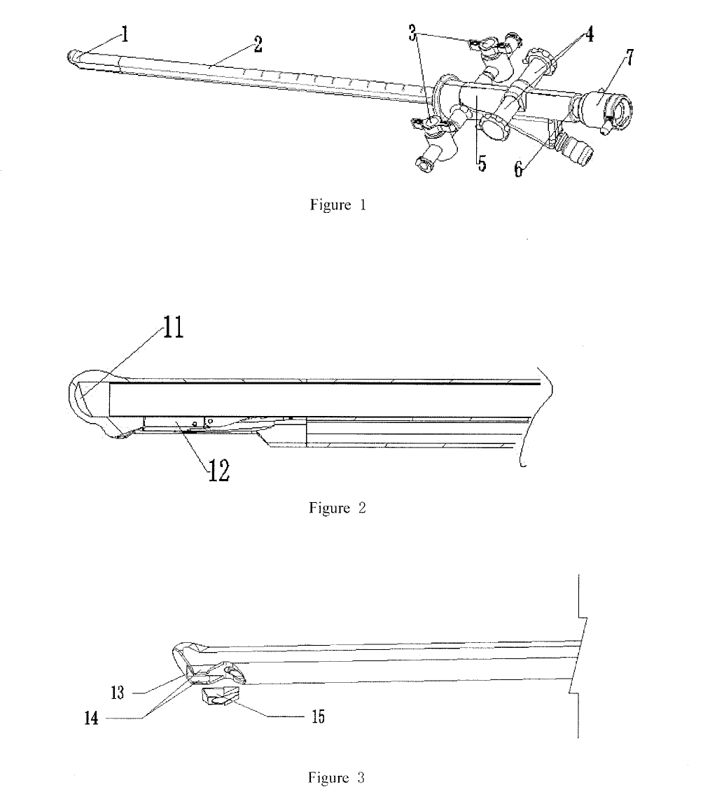

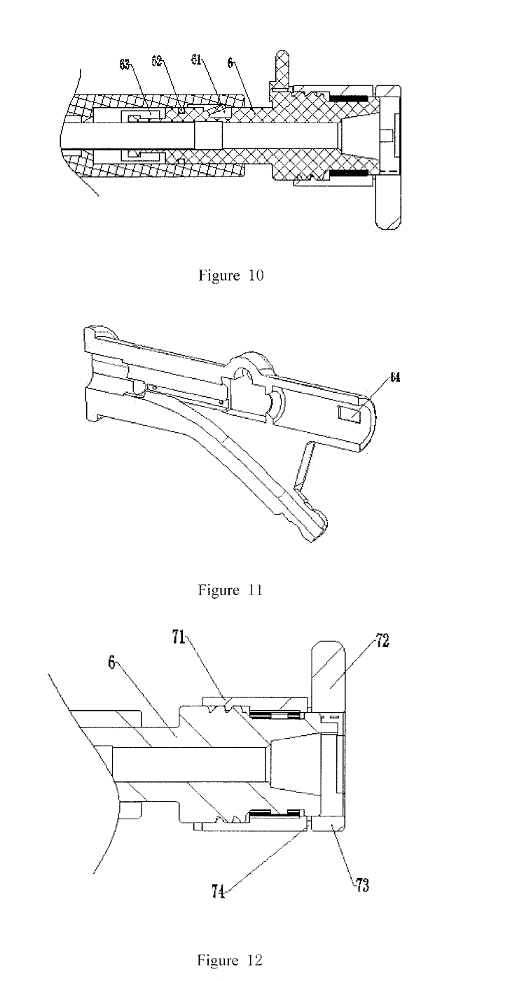

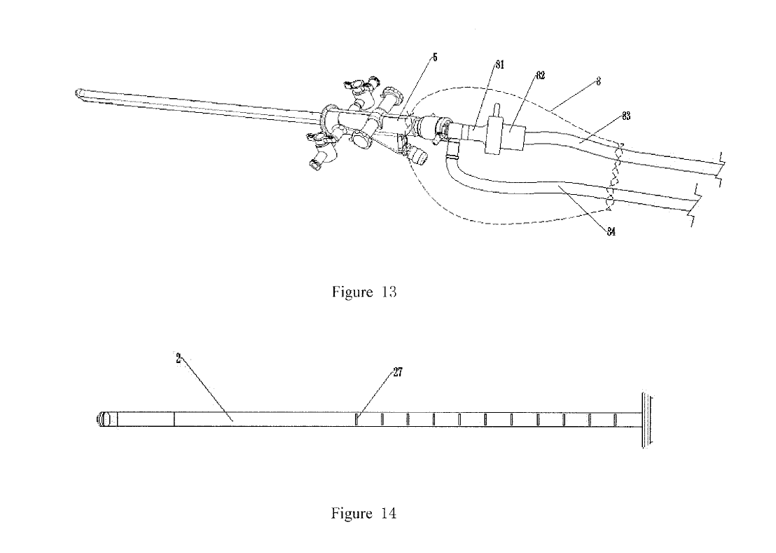

As shown in FIGS. 1-14, the disposable endoscope sheath comprises an insertion portion, a control portion, an endoscope locking portion; wherein, the insertion portion comprises a sheath head 1 and a sheath tube 2; the control portion comprises a main body 5, a water inlet and outlet valve 3 and an instrument adjusting knob 4; the endoscope locking portion comprises an extension endoscope bridge 6 and a locking sleeve 7; the sheath head is located at the front end of the insertion portion and is connected with the sheath tube in the rear; the sheath tube is connected with the extension endoscope bridge through the main body; the water inlet and outlet valve and the instrument adjusting knob are located on the main body of the endoscope sheath; the lower part of the front end of the sheath tube is provided with a lifting device 12; the sheath head is provided with an arc-shaped transparent widow 11; the sheath tube comprises an endoscope cavity 21, an instrument channel 22, a water inflow and outflow channel 24 and a steel wire channel 25. The lower part of the front end of the endoscope cavity is provided with an inserting groove 13, flanges 14 on two sides of the inserting groove and a stopper which is not on the same side with the inserting groove; the inserting groove and the flanges are formed into a replacement groove for replacing the projection; the inserting groove is internally provided with a replaceable projection 15; the instrument adjusting knob comprises a rack 41 and a gear 42; the rack and the gear are engaged; one end of the steel wire 43 is fixed on the rack, while the other end is connected with the lifting device via the steel wire channel. The extension endoscope bridge comprises an endoscope bridge, a rebound blade 61, a rubber ring 62, a rubber sleeve 63 and a groove-shaped space 64. The locking sleeve comprises an endoscope bridge interface, an endoscope locking sleeve 71, a locking lifting lever 72, a locking compression ring 73 and a disposable connecting ring connected to the tail end of the endoscope bridge. The inner hole of the endoscope locking sleeve is provided with a movable one-way gear; the movable one-way gear is provided with screw threads at the rear end; the exterior of the tail end of the endoscope bridge is also provided with one-way gear and screw threads in turn, matched with the movable gear of the inner hole of the endoscope locking sleeve and connected with the endoscope locking sleeve in a threaded way. The inner hole of the endoscope bridge interface at the tail end of the endoscope bridge is of a conical design adapted to the locking interface of the endoscope. The rear end portion of the endoscope sheath is also provided with a protective cover 8. The endoscope cavity of the sheath tube is internally sleeved with a metal tube 26, and the outer surface of said insertion portion has scale marks 27.

A Working process of this embodiment: The design of the extension endoscope bridge ensures that the use of this endoscope sheath is not limited by the length of the endoscope. The extension endoscope bridge is usually made from medical plastic, with a certain rebound force. When the endoscope is inserted, the rebound blade is deformed, and when the internal groove-shaped space gets bigger, springs back automatically. The rebound blade springs back to clamp the main section which cannot be pulled out, ensuring that the endoscope does not fall when pulled back. The rubber ring is used for sealing the endoscope bridge and the main body. The metal rube and the endoscope sheath of plastic material form clearance fit. Meanwhile, a silicone rubber sleeve with good elasticity is disposed between the endoscope sheath and the metal tube, ensuring the air tightness between the metal tube and the endoscope sheath. The endoscope sheath itself has a certain depth, thereby forming a reciprocating axial movement space.

The endoscope locking sleeve, the locking lifting lever, the locking compression ring and the disposable connecting ring are initially integrated. The endoscope locking sleeve and the locking compression ring are connected by the disposable connecting ring. A disposable locking cracking slot 74 is formed next to the disposable connecting ring. By such design, the endoscope is conveniently installed at the endoscope locking sleeve in the rear of the endoscope sheath, and after use, the disposable connecting ring is torn off along the disposable locking cracking slot such that the endoscope locking sleeve and the locking compression ring are separated, and then the endoscope can be conveniently taken out of the endoscope cavity and the endoscope bridge of the endoscope sheath. The locking lifting lever on the exterior of the locking compressor ring drives the locking compression ring to rotate, such that the clamping plate of the locking compression ring is locked into the locking groove of the endoscope. The inner hole of the endoscope locking sleeve is provided with a movable one-way gear; the movable one-way gear is provided with screw threads at the rear end; the exterior of the tail end of the endoscope bridge is also provided with one-way gear and screw threads in turn, matched with the movable gear of the inner hole of the endoscope locking sleeve and connected with the endoscope locking sleeve in a threaded way. The two are rotated and then can be locked in placed, playing the role of locking and fixing the endoscope. The one-way gear of the endoscope locking sleeve and the one-way gear on the exterior of the tail end of the endoscope bridge are matched with each other, ensuring that the locking ring in connection with the endoscope locking sleeve can only rotate and lock the endoscope in one direction and cannot move back to unlock the endoscope. The disposable connecting ring must be torn off to separate the endoscope locking sleeve and the locking compression ring, and then the endoscope can be taken out of the endoscope cavity and the endoscope bridge of the endoscope sheath, ensuring that the endoscope sheath can be used at one time; the inner hole of the endoscope bridge interface at the tail end of the endoscope bridge is of a conical design adapted to the locking interface of the endoscope, ensuring that the endoscope sheath is sealed without leakage during surgery.

The fixed projection forces the surgical instrument to form a fixed angle. On the other hand, by changing the replaceable projections capable of changing the angles, the angle between the surgical instrument and the tube sheath can be changed, meeting the demands on different instrument angles. The replaceable projection is inserted into the dovetail groove. In such circumstances, with a little force, the replaceable projection has a small deformation during the insertion. With further thrust on the replaceable projection until the projection is pushed to the position of the stopper along with the friction force of the plastic, the fixing is complete. At this time, the replaceable projection is always protected by the flanges, dovetail groove and stopper during insertion and back movement in the surgery, and does not fall. Thereby, the safety of the product is ensured.

In the endoscopic surgery, the angle of the surgical instrument is needed to be adjusted sometimes. In such circumstances, a device capable of optionally adjusting the instrument angle is needed. Therefore, this endoscope sheath includes a set of instrument angle adjusting structure. The instrument adjusting knob on the control portion is rotated to drive the internal gear to rotate. In such conditions, the gear drives the rack and also drives the steel wire on the rack. The steel wire is connected with the lifting device via the steel wire channel, driving the lifting device to swing at a certain angle. The lifting device and the inserted surgical instrument are in close fit, thereby driving the surgical instrument to swing at a certain angle, realizing the adjustment to the angle of the surgical instrument in the instrument channel. Here, the gears and racks involved are both made from medical plastic with high hardness, ensuring the durability of the instrument.

In some endoscopy, the medical cold light source used, so the endoscope front end is provided with an arc-shaped transparent window, and the medical plastic with high transparency is employed. The interior of the window is of a smooth design, and the central angle is an acute angle. Such design can minimize the effects of light reflection on the observation by using the endoscope and avoid the phenomenon that the endoscope is unclear due to the light reflection during observation.

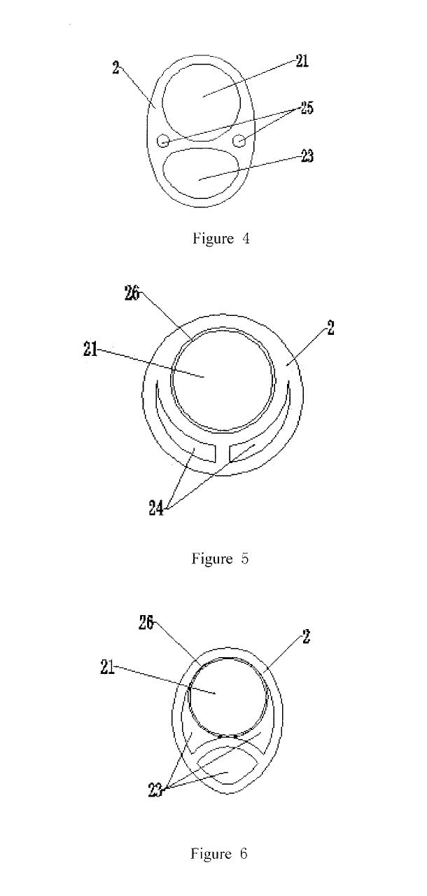

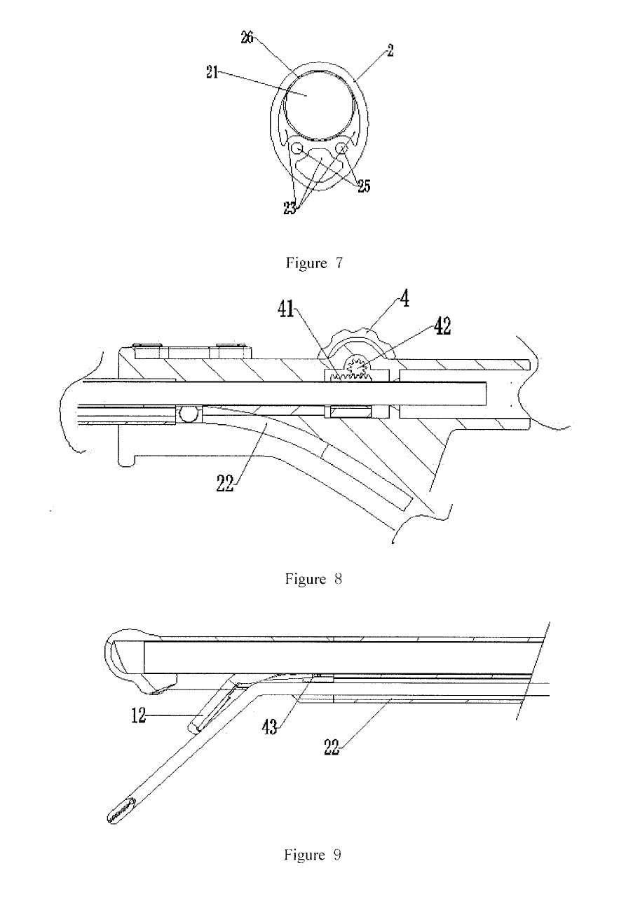

In a usual surgery, the sheath tube with the cross section as shown in FIG. 4 is used. The endoscope enters a human body via the endoscope channel; the surgical instrument enters the human body via the instrument channel and the water inflow and outflow channel; and the channel can also control the inflow and outflow water via an external valve to rinse the human body. The steel wire controlling the angle of the surgical instrument can control the surgical instrument via the steel wire channel; and if only the endoscopy is needed while surgery is not needed, the sheath tube with the cross section can be seen in FIG. 5 can be selected. The endoscope enters the human body via the endoscope channel. The water inflow and outflow channel can control the inflow and outflow water via the water inlet and outlet valve to rinse the human body. Meanwhile, the endoscope channel is also provided with a metal tube for increasing the strength of the endoscope channel. Sometimes, in the endoscopic surgery, more than one surgical instrument is needed. In such circumstances, the sheath tube with the cross section as shown in FIG. 6 can be selected. The endoscope enters the human body via the endoscope channel. A plurality of instruments can enter the human via the water inlet and outlet channel and instrument channel (23). At the same time, such channel can also control the inflow and outflow water through the water inlet and outlet valve to rinse human body. Sometimes, during the endoscopic surgery, more than one surgical instruments are needed, and the steel wire is also required to control a certain instrument. In such circumstances, the sheath tube with the cross section as shown in FIG. 7 can be selected. The endoscope enters the human body via the endoscope channel. A plurality of instruments can enter the human via the water inlet and outlet channel and instrument channel. At the same time, such channel can also control the inflow and outflow water through the water inlet and outlet valve to rinse human body. The steel wire can control the surgical instrument via the steel wire channel.

The insertion portion is provided with scale marks, and the scale marks are disposed from the rear end of the sheath head to the main body of the control portion. If the scale marks start from the top of the insertion portion, there is a certain surgical depth in the endoscopic surgery, and then the scales on the top are wasted, so the scale marks are formed from the rear end of the sheath head, thereby primarily judging the depth scope of the focus and further judging the disease.

In some endoscopic surgeries, the cases that a lot of body fluid injects out cannot be predicted, for example, hemorrhoea, so before the endoscopic surgery, the endoscope can be placed in the endoscope sheath. The endoscope rear end 81 is connected with the CCD system interface 82, while the optical cable and the video cable are in good connection conditions. One end of the protective cover is connected to the rear end of the protective cover is connected to the rear end of the endoscope, wrapping up the rear end of the endoscope sheath; the other end wraps up the video cable 83 and the optical cable 84, ensuring that the endoscope rear end and the CCD system interface are not polluted when the a lot of body fluid injects during the surgery. Here, the protective cover is made from medical watertight materials.

The above are detailed descriptions of one embodiment of the present invention. However, the contents are a preferable embodiment of the present invention and cannot be regarded as limiting the implementation scope of the present invention. All equivalent changes and improvements made on the basis of application scope of the present invention shall fall within the protective scope of the present invention.

What is claimed is:

1. A disposable endoscope sheath, comprising an insertion portion, a control portion and an endoscope locking portion; wherein the control portion comprises a sheath head and a sheath tube; the control portion comprises a main body, and a water inlet and outlet valve; the endoscope locking portion comprises an extension endoscope bridge and a locking sleeve; the sheath head is located at the front end of the insertion portion and is connected with the sheath tube in the rear; the sheath tube is connected with the extension endoscope bridge of the endoscope locking portion; the water inlet and outlet valve and an instrument adjusting knob are located on the main body of the endoscope sheath; characterized in that, the sheath tube comprises an endoscope cavity, an instrument channel, a steel wire channel and a water inflow and outflow channel; the lower part of the front end of the sheath tube is provided with a surgical instrument angle adjusting device, and this device may be a fixed projection, a lifting device or a replaceable projection.

2. The disposable endoscope sheath according to claim 1, characterized in that, said locking sleeve comprises an endoscope bridge interface, an endoscope locking sleeve, a locking lifting lever, a locking compression ring and a disposal connecting ring connected to the tail end of the endoscope bridge; the endoscope locking sleeve and the locking compression ring are integrally connected by using a disposable connecting ring; the exterior of the locking compression ring is connected with the locking lifting lever; the shape of the inner hole of the clamping plate of the locking compression ring corresponds to that of the locking plate of the locking interface of the endoscope; the size of the clamping plate of the locking compression ring is matched with a locking groove of the locking interface of the endoscope; an inner hole of the endoscope locking sleeve is provided with a movable one-way gear; the movable one-way gear is provided with screw threads at the rear end; the exterior of the tail end of the endoscope bridge is also provided with one-way gear and screw threads in turn, matched with the movable gear of the inner hole of the endoscope locking sleeve and connected with the endoscope locking sleeve in a threaded way.

3. The disposable endoscope sheath according to claim 1, characterized in that, the lower part of the front end of said endoscope cavity is provided with an inserting groove, flanges on two sides of the inserting groove and a stopper which is not on the same side with the flanges; the inserting groove is a dovetail groove; the dovetail groove is a trapezoidal dovetail structure; and the inserting groove and the flanges are formed into a replacement groove for replacing the projection.

4. The disposable endoscope sheath according to claim 1, also comprising an instrument adjusting knob; the instrument adjusting knob comprising a gear and a rack; the gear and the rack being engaged; one end of a steel wire is fixed on the rack, while the other end being connected with the lifting device via a steel wire channel.

5. The disposable endoscope sheath according to claim 1, characterized in that, the extension endoscope bridge comprises an endoscope bridge, a rebound blade and a groove-shaped space; the rebound blade is disposed inside the endoscope bridge; and the groove-shaped space is located at the rear end of the endoscope sheath.

6. The disposable endoscope sheath according to claim 1, characterized in that, the sheath head is provided with an arc-shaped transparent window; the inner surface of the arc-shaped transparent window is designed to be smooth, and the corresponding central angle is an acute angle.

7. The disposable endoscope sheath according to claim 1, characterized in that, the endoscope cavity of the sheath tube is internally sleeved with a metal tube.

8. The disposable endoscope sheath according to claim 2, characterized in that, the inner hole of the endoscope bridge interface at the tail end of the endoscope bridge is of a conical design adapted to the locking interface of the endoscope.

9. The disposable endoscope sheath according to claim 1, characterized in that, the rear end portion of the endoscope sheath is also provided with a protective cover; one end of said protective cover wraps up the rear end of the main body of the control portion of the endoscope sheath, while the other end wraps up a cable of a CCD observation system.

10. The disposable endoscope sheath according to claim 1, characterized in that, the outer surface of said insertion portion has scale marks; and the scale marks are disposed from the rear end of the sheath head to the main body of the control portion.

11. The disposable endoscope sheath according to claim 4, characterized in that, the materials of said gear and rack are medical plastic materials with high hardness.

12. The disposable endoscope sheath according to claim 5, characterized in that, said extension endoscope bridge also comprises a rubber ring and a rubber sleeve; and said rubber ring and rubber sleeve are made from silica gel.

13. The disposable endoscope sheath according to claim 9, characterized in that, the material of the protective cover is medical watertight plastic.
